(19) United States
(12) Patent Application Publication
(10) Pub. No.: US 2015/0174083 A1
(43) Pub. Date: Jun. 25, 2015

(54) USE OF FLUOXETINE FOR INCREASING MEAT AND MILK PRODUCTION

(71) Applicant: Sanovel Hayvan Sagligi Urunleri Sanayi Ve Ticaret Anonim Sirketi, Istanbul (TR)

(72) Inventors: Umit Cifter, Istanbul (TR); Ali Turkyilmaz, Istanbul (TR); Onur Mutlu, Istanbul (TR)

(21) Appl. No.: 14/418,757

(22) PCT Filed: Jul. 29, 2013
(86) PCT No.: PCT/TR2013/000246

§ 371 Date: Jan. 30, 2015

(30) Foreign Application Priority Data
Jul. 31, 2012 (TR) 2012/08872
Sep. 5, 2012 (TR) 2012/10112
Sep. 6, 2012 (TR) 2012/10187

(51) Int. Cl. A61K 31/138; A61K 31/381; A61K 47/14; A61K 47/22; A61K 47/02; A61K 31/5513; A61K 47/10

(52) U.S. Cl. CPC A61K 31/138; A61K 31/5513; A61K 31/381; A61K 47/10; A61K 47/22; A61K 47/02; A61K 47/14

(57) ABSTRACT

The present invention relates to a method for increasing meat and milk production in livestock, wherein fluoxetine or a pharmaceutically acceptable salt, solvate, polymorph, or a racemic mixture thereof is administered to the livestock.

### FIELD OF INVENTION

This invention relates to the use of fluoxetine or a pharmaceutically acceptable salt thereof for increasing meat and milk production in livestock.

### BACKGROUND OF INVENTION

Nowadays, with the world's population reaching 7 billions, the most pronounced problem of the humans is meeting the nutritional needs. Especially the need for meat and milk products with protein content is increasing day by day. Protein-based foods constitute the most valuable and prominent part of nutrition and the need for protein is increasing day by day. Despite the fact that some of the protein production is obtained from plants, it is mainly derived from animal-based resources. Although the nutritional needs are increasing in line with the increasing world population; water, fossil fuel and cereal resources used in breeding livestock are decreasing. These resources should be used more efficiently. Additionally, increasing the milk and meat efficiency of livestock makes up the most significant dimension of the solution.

Fluoxetine, with the chemical name (+/−)-N-methyl-3-phenyl-3-(alpha,alpha,alpha-trifluoro-p-tolyloxy)propylamine, is a prototype of the selective serotonin reuptake inhibitors (SSRI), and has a half life which is longer than those of the other members in this group. It is used as an antidepressant. Its chemical structure is illustrated with Formula I given below.

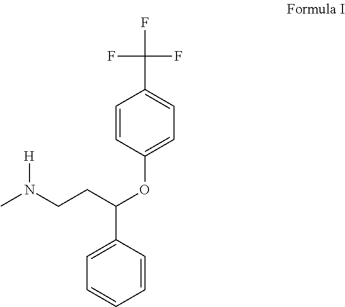 [SMILES: CNCCC(Oc1ccc(C(F)(F)F)cc1)c1ccccc1]

The fluoxetine molecule was disclosed in the patent DE2500110 for the first time.

The patent application EP0123469 discloses the use of fluoxetine or norfluoxetine in the treatment of anxiety.

The patent application EP0294028 discloses the use of fluoxetine in the treatment of diabetes without inducing weight loss.

When the increasing food requirements are considered, the vital importance can be seen of augmenting the protein-containing animal-based foodstuffs and increasing the production efficiency. The use of fluoxetine for these purposes has not been disclosed in any other documents so far.

Considering these problems and needs, it becomes obvious that a novelty is required in the technical field related to augmenting meat and milk efficiency.

### OBJECT AND BRIEF DESCRIPTION OF INVENTION

The present invention relates to the use of fluoxetine, eliminating all aforesaid problems and bringing additional advantages to the relevant prior art.

Accordingly, the main object of the present invention is to increase the meat and milk production from livestock by means of a novel use of fluoxetine.

Another object of the present invention is to stimulate appetite in livestock by means of a novel use of fluoxetine.

A further object of the present invention is to stimulate hyperlipidemia, increased fat, and fat storage in livestock by means of a novel use of fluoxetine.

Another object of the present invention is to stimulate an increase in the prolactin and bovine somatotropin hormones in livestock by means of a novel use of fluoxetine.

A further object of the present invention is to increase the meat and milk production from livestock by means of a novel use of a stable formulation of fluoxetine.

Another object of the present invention is to increase the meat and milk production from livestock by means of a novel use of a stable injectable formulation of fluoxetine.

A further object of the present invention is to suppress the libido in livestock by means of a novel use of an injectable stable formulation of fluoxetine.

A method for increasing meat and milk production in livestock has been developed to achieve all objects, referred to above and to emerge from the following detailed disclosure.

According to a preferred embodiment of the present invention, said novel method comprises administering fluoxetine or a pharmaceutically acceptable salt, solvate, polymorph, or a racemic mixture thereof to the livestock.

According to a preferred embodiment of the present invention, said novel method comprises administering a formulation containing fluoxetine or a pharmaceutically acceptable salt, solvate, polymorph, or a racemic mixture thereof to the livestock.

According to a preferred embodiment of the present invention, said novel method comprises administering an injectable formulation containing fluoxetine or a pharmaceutically acceptable salt, solvate, polymorph, or a racemic mixture thereof to the livestock.

According to a preferred embodiment of the present invention, said novel method comprises administering a lipid-based injectable formulation containing fluoxetine or a pharmaceutically acceptable salt, solvate, polymorph, or a racemic mixture thereof to the livestock.

According to a preferred embodiment of the present invention, the formulation administered to the livestock according to said method also comprises one or a mixture of both of olanzapine and/or duloxetine in a pharmaceutically acceptable amount.

According to a preferred embodiment of the present invention, the formulation administered to the livestock according to said method comprises fluoxetine in an amount of 0.05 to 0.4 mg/kgca/day.

According to another preferred embodiment of the present invention, the formulation administered to the livestock according to said method comprises olanzapine in an amount of 0.05 to 0.4 mg/kgca/day.

According to a preferred embodiment of the present invention, the formulation administered to the livestock according to said method comprises duloxetine in an amount of 0.05 to 0.4 mg/kgca/day. Here the expression “mg/kgca/day” means “milligram/kilogram of live animal/day”.

According to another preferred embodiment of the present invention, the formulation administered to the livestock according to said method comprises polyethylene glycol as a solvent.

According to another preferred embodiment of the present invention, the formulation administered to the livestock according to said method comprises alpha tocopherol as an antioxidant.

According to another preferred embodiment of the present invention, the formulation administered to the livestock according to said method comprises sodium hydroxide or hydrochloric acid as a pH regulator.

According to another preferred embodiment of the present invention, the formulation administered to the livestock according to said method comprises methylparaben as an antimicrobial agent.

### DETAILED DESCRIPTION OF INVENTION

### Example 1

a. 0.5-10% by weight of fluoxetine,
b. 20-99% by weight of polyethylene glycol (solvent),
c. 0.05-0.075% by weight of alpha tocopherol (antioxidant),
d. 0.5-5% by weight of NaOH/HCl (pH regulator),
e. 0.05-0.18% by weight of methylparaben (antimicrobial agent).

### Preparation Method 1

Alpha tocopherol and methylparaben are dissolved in polyethylene glycol, previously heated to 50-80° C., and then cooled down. Then, fluoxetine is added thereto and dispersed homogenously. The pH thereof is regulated using NaOH/HCl, and then filtered. Following sterilization, it is filled into vials or alternatively, sterilization is performed after filling is made into vials.

### Preparation Method 2

Alpha tocopherol, methylparaben, and fluoxetine are suspended in polyethylene glycol. The pH thereof is regulated using NaOH/HCl, cooled, and then filtered. Following sterilization, it is filled into vials or alternatively, sterilization is performed after filling is made into vials.

### Example 2

a. 0.5-10% by weight of fluoxetine or duloxetine,
b. 0.5-30% by weight of olanzapine,
c. 20-99% by weight of polyethylene glycol (solvent),
d. 0.05-0.075% by weight of alpha tocopherol (antioxidant),
e. 0.5-5% by weight of NaOH/HCl (pH regulator),
f. 0.05-0.18% by weight of methylparaben (antimicrobial agent).

### Preparation Method 1

Alpha tocopherol and methylparaben are dissolved in polyethylene glycol, previously heated to 50-80° C., and then cooled down. Then, olanzapine plus duloxetine or fluoxetine are added thereto and dispersed homogenously. The pH thereof is regulated using NaOH/HCl, and then filtered. Following sterilization, it is filled into vials or alternatively, sterilization is performed after filling is made into vials.

### Preparation Method 2

Alpha tocopherol, methylparaben, and fluoxetine plus olanzapine or duloxetine are suspended in polyethylene glycol. The pH thereof is regulated using NaOH/HCl, cooled, and then filtered. Following sterilization, it is filled into vials or alternatively, sterilization is performed after filling is made into vials.

### Example 3

a. 0.5-10% by weight of fluoxetine,
b. 20-99% by weight of sesame oil (solvent),
c. 0.05-0.075% by weight of alpha tocopherol (antioxidant).

### Preparation Method

A sterile lyophilized powder of fluoxetine and alpha tocopherol is prepared in vials. Before use, it is reconstituted with sterile water or sesame oil and is injected intramuscularly.

### Example 4

a. 0.5-30% by weight of olanzapine,
b. 0.5-10% by weight of duloxetine or fluoxetine,
c. 20-99% by weight of sterile water or sesame oil (solvent),
d. 0.05-0.075% by weight of alpha tocopherol (antioxidant).

### Preparation Method

A sterile lyophilized powder of olanzapine plus duloxetine or fluoxetine and alpha tocopherol is prepared in vials. Before use, it is reconstituted with sterile water or sesame oil and is injected intramuscularly.

The lipid-based formulations in the examples above may be long acting.

### Alternative Formulation Types

1. These formulations can be prepared in the form of aqueous or oily solutions. Since olanzapine is not dissolved in water, a co-solvent should be used. The carrier agents used in oily solutions can be sesame oil, cotton oil, peanut oil, and opium oil.
2. Reconstitutable systems can be prepared. Nanoparticles, sterile powder fill and freeze-drying (lyophilization) systems can be prepared.
3. These formulations may be present in a suspension form. The active agent is not dissolved, but dispersed in the liquid carrier.
4. Liposome and emulsions can be prepared. Oil/water or water/oil or oil/water/oil emulsions can be prepared using convenient surface active agents.

With the present invention, the meat and milk production in livestock can be surprisingly increased by making use of fluoxetine. Said formulation also comprises olanzapine or duloxetine or the both at the same time. The libido can also be suppressed in the livestock. The formulations according to the present invention feature high stability, high solubility, and high dissolution rates, and are used preferably in an injectable form. With the method according to the present invention, the livestock show a surprisingly increased appetite, hyperlipidemia and increased fat, increased fat storage, and increased prolactin hormone and bovine somatotropin. The level of the testosterone hormone is reduced in male livestock. The injectable solution is administered in an amount of 10 ml and preferably 5 ml. Thus, undesired outcomes such as abscesses and local reactions are prevented in the application site. Alpha tocopherol is particularly preferred in the formulations according to the present invention, because alpha tocopherol provides better stability than other antioxidants do. Additionally, the miscibility and uniform distribution of those components composing the solution are increased.

The livestock are cattle, sheep, goats, rabbits, poultry, and swine.

The pharmaceutical formulations according to the present invention may also comprise one or more pharmaceutically acceptable excipient(s). Pharmaceutically acceptable excipients include, but are not restricted to mass increasing agents, surface stabilizers, carriers/solvents, co-solvents (used to prepare aqueous systems for active agents not dissolvable in water), etc. and the mixtures thereof.

Suitable mass increasing agents include, but are not restricted to mannitol, lactose, sucrose, and dextran.

Suitable surface stabilizers (suspending agents, carrier agents) (0.5-99%, 0.1-50%) include, but are not restricted to low molecular weight oligomers, surfactants, polysorbate 80, benzalkonium chloride, low viscosity hydroxypropyl cellulose (HPC or HPC-SL), HPMC, HMC, ethyl cellulose, povidone, pluronics, sodium deoxycholate, peg-phospholipids, tyloxapol and other tritones, PVP, SLS, dioctyl sulfosuccinate, gelatin, casein, lecithin, dextran, acacia gum, stearic acid, calcium stearate, glycerol monostearate, sorbitan esters, polyoxyethylene alkyl ethers, polyethylene glycols, triethanolamine, polyvinyl alcohol, poloxamers (pluronic f68, f108), poloxamines (tetronic 908, poloxamine 908), cationic agents (methyltrioctylammonium chloride (aliquat 336), tetrabutylammonium bromide, choline esters).

Suitable carriers/solvents include, but are not restricted to water, alcohol, and oil.

Suitable co-solvents are used for preparing aqueous systems of active agents not dissolvable in water, and include, but are not restricted to
liquid co-solvents: glycerin, PEG (300, 400, 3350), propylene alcohol, ethanol, Cremophor EL, Sorbitol;
surface active agents: Polysorbate 80, 20, Pluronic 68, lecithin;
complex agents: β-cyclodextrin, PVP, NaCMC.

Suitable antimicrobial agents include, but are not restricted to phenol, m-cresol, methylparaben, propylparaben, chlorobutanol, benzyl alcohol, benzalkonium chloride, thimerosal.

Suitable antioxidant agents include, but are not restricted to sodium bisulfite, sodium sulfite, sodium metabisulfite, sodium thiosulphate, sodium formaldehyde, ascorbic acid isomers, acetylcysteine, cysteine, thioglycerol, thioglycolic acid, thiolactic acid, thiourea, glutathione, propyl gallate, butylated hydroxyanisole, butylated hydroxytoluene, ascorbyl palmitate, α-tocopherol.

Suitable pH regulators/buffering agents include, but are not restricted to acetic acid/acetate, citric acid/citrate, phosphoric acid/phosphate, glutamic acid/glutamate.

1. A method for increasing meat and milk production in livestock, wherein fluoxetine or a pharmaceutically acceptable salt, solvate, polymorph, or a racemic mixture thereof is administered to the livestock.

2. A method according to claim 1, wherein a formulation comprising fluoxetine or a pharmaceutically acceptable salt, solvate, polymorph, or a racemic mixture thereof is administered to the livestock.

3. A method according to claim 1, wherein an injectable formulation comprising fluoxetine or a pharmaceutically acceptable salt, solvate, polymorph, or a racemic mixture thereof is administered to the livestock.

4. A method according to claim 1, wherein a lipid-based injectable formulation comprising fluoxetine or a pharmaceutically acceptable salt, solvate, polymorph, or a racemic mixture thereof is administered to the livestock.

5. A method according to claim 1, wherein the formulation administered to the livestock comprises one or a mixture of both of olanzapine and/or duloxetine in a pharmaceutically acceptable amount.

6. A method according to claim 1, wherein said injectable solution is administered in an amount of 10 ml and preferably in an amount of 5 ml.

7. A method according to claim 1, wherein the formulation administered to the livestock comprises fluoxetine in an amount of 0.05-0.4 mg/kgca/day.

8. A method according to claim 1, wherein the formulation administered to the livestock comprises olanzapine in an amount of 0.05-0.4 mg/kgca/day.

9. A method according to claim 1, wherein the formulation administered to the livestock comprises duloxetine in an amount of 0.05-0.4 mg/kgca/day.

10. A method according to claim 1, wherein the formulation administered to the livestock consist of,
a. 0.5-10% by weight of fluoxetine,
b. 20-99% by weight of polyethylene glycol (solvent),
c. 0.05-0.075% by weight of alpha tocopherol (antioxidant),
d. 0.5-5% by weight of NaOH/HCl (pH regulator),
e. 0.05-0.18% by weight of methylparaben (antimicrobial agent).

11. A method according to claim 1, wherein the formulation administered to the livestock consist of,
a. 0.5-10% by weight of duloxetine or fluoxetine,
b. 0.5-30% by weight of olanzapine,
c. 20-99% by weight of polyethylene glycol (solvent),
d. 0.05-0.075% by weight of alpha tocopherol (antioxidant),
e. 0.5-5% by weight of NaOH/HCl (pH regulator),
f. 0.05-0.18% by weight of methylparaben (antimicrobial agent).

12. A method according to claim 1, wherein the formulation administered to the livestock consist of,
a. 0.5-30% by weight of fluoxetine,
b. 20-99% by weight of sesame oil (solvent),
c. 0.05-0.075% by weight of alpha tocopherol (antioxidant).

13. A method according to claim 1, wherein the formulation administered to the livestock consist of
a. 0.5-30% by weight of olanzapine,
b. 0.5-10% by weight of duloxetine or fluoxetine,
c. 20-99% by weight of sterile water or sesame oil (solvent),
d. 0.05-0.075% by weight of alpha tocopherol (antioxidant).

14. A method according to claim 1, wherein the formulation administered to the livestock comprises alpha tocopherol as an antioxidant.
